(19) United States
(12) Patent Application Publication
(10) Pub. No.: US 2025/0116654 A1
(43) Pub. Date: Apr. 10, 2025

(54) AIRCRAFT GALLEY INSERT WITH PATHOGENS DIAGNOSTIC TEST CAPABILITIES

(71) Applicant: B/E Aerospace, Inc., Winston Salem, NC (US)

(72) Inventors: Arnau Castillo Gonzalez, Maarssen (NL); Antonio Martinez Murcia, Elche (ES)

(73) Assignee: B/E Aerospace, Inc., Winston Salem, NC (US)

(21) Appl. No.: 18/982,817

(22) Filed: Dec. 16, 2024

Related U.S. Application Data
(62) Division of application No. 17/518,339, filed on Nov. 3, 2021.
(60) Provisional application No. 63/114,330, filed on Nov. 16, 2020.
(60) Provisional application No. 63/114,339, filed on Nov. 16, 2020.
(60) Provisional application No. 63/114,350, filed on Nov. 16, 2020.
(60) Provisional application No. 63/114,064, filed on Nov. 16, 2020.
(60) Provisional application No. 63/114,366, filed on Nov. 16, 2020.
(60) Provisional application No. 63/114,157, filed on Nov. 16, 2020.
(60) Provisional application No. 63/114,386, filed on Nov. 16, 2020.
(60) Provisional application No. 63/114,400, filed on Nov. 16, 2020.

(51) Int. Cl. B64D 11/04; B01L 3/02; B01L 7/00; B64D 11/00; C12Q 1/686

(52) U.S. Cl. CPC G01N 33/497; B01L 1/00; B01L 3/021; B01L 3/505; B01L 7/52; B64D 13/08; C12Q 1/04; C12Q 1/686; C12Q 1/70; G01N 1/10; G01N 1/2226; G01N 1/2247; G01N 1/40; G16B 10/00; G16B 40/00; G16B 50/10; B01L 2200/028; B01L 2200/10; B01L 2200/18; B01L 2300/023; B01L 2300/18; B64D 13/06; B64D 2013/0603; G01N 2001/1031; G01N 1/2205; G01N 2001/2244; G01N 33/4975; H04L 67/12

(57) ABSTRACT

A modular aircraft galley insert for performing thermal cycling for PCR testing includes a housing having a sample module for receiving a sample to be tested, a thermal module thermally connected to the sample module for cycling temperatures of the sample to be tested, a power module for providing power to the thermal module, a detector module configured to analyze the sample and connected to receive power from the power module, and a connectivity module for communicating results of a test from the detector module to a remote location with respect to the housing.

### CROSS-REFERENCE TO RELATED APPLICATIONS

This application is a divisional of, claims priority to and the benefit of, U.S. patent application Ser. No. 17/518,339, filed Nov. 3, 2021, entitled “AIRCRAFT GALLEY INSERT WITH PATHOGENS DIAGNOSTIC TEST CAPABILITIES.” The '339 application is a non-provisional of U.S. Provisional Patent Applications with the following Ser. Nos. 63/114,330, 63/114,339, 63/114,350, 63/114,400, 63/114,064, 63/114,157, 63/114,386, 63/114,366 all filed on Nov. 16, 2020. All of which are incorporated by reference herein in their entirety for all purposes.

### FIELD

The present application is related to a system and method used to collect a representative air sample of an aircraft, more specifically to a method, and systems for analyzing a biological sample on an aircraft through a pathogen/contaminant diagnostic test such as real time polymerase chain reaction (PCR) test.

### BACKGROUND

The spread progression of SARS-CoV-2 around the world has risen a red flag: Economic globalization creates systemic risks. As trade, finance, travel, cyber and other networks grow in scale and interact, they become more complex and unstable. The transporters of the goods of the global economy, such as major airport hubs, are also spreaders of the pathogens. The 2008 global financial crisis provided a dramatic example of how contagions could spread from the US to global markets overnight. So too has the rapid spread of cyber viruses. In health, rising life expectancy and success in preventing a repeat of the devastating influenza pandemic of 1918, which infected about one-third of the world's population and killed as many as 50 m people, has created a false sense of security. But the world is now more interdependent. For example, China represents almost one-fifth of global output, is integral to global supply chains, and its tourists spend over $260 billion annually. The COVID-19 pandemic shed light on the need for better monitoring, detecting, and isolating ill passengers, specifically due to the havoc that was wreaked detrimental impact on the global economy, specifically air travel due to closed borders, movement restrictions, and testing requirements.

However, the COVID-19 pandemic the air travel industry has proven that air travel can be safe and that aircraft cabins have a well-managed airflow that inhibits minimize the risk for transmission of virus, and that being seated onboard an aircraft is safer than shopping in large stores. Governments and other authorities need to assume that aircraft are contaminated until proven “clean.” as 25% of COVID-19 cases are asymptomatic or pre-symptomatic; but still contagious. Thus, if borders shutdown and a drastic reduction in international travel global passenger travel is greatly reduced. To date travelers and governments have relied on individual diagnostic tests. The uncertainty of the results has reduced people's inclination to travel and subsequent airline inclination to maintain routes.

Accordingly, conventional systems and methods of monitoring infections has not lived up to requirements of the fast-paced modern world. Thus, there is still a need in the art for an improved on-board virus and pathogen detection system. The present disclosure provides a solution for this need.

### SUMMARY

A modular aircraft galley insert for performing pathogen/contaminant diagnostic testing such as thermal cycling for PCR testing is disclosed. The insert includes a housing having, a sample module for receiving a sample to be tested, a sample concentration module, a purification and extraction module, a thermal module thermally connected to the sample module for cycling temperatures of the sample to be tested, a power module for providing power to the different modules, a detector module configured to analyze the amplified signal obtained from the sample and connected to receive power from the power module, the signal should be detected after each amplification cycle, and a connectivity module for communicating results of a test from the detector module to a remote location with respect to the housing.

A storage space can be located within the housing for storing for consumables and process waste. The waste generated is not medical waste. The consumables are inert, water based, and safe for the environmental. The samples do not contain human biological information (DNA/RNA). A second storage space can be present for storing the tested sample. A module for sample automatic loading. This module is configured to transfer a portion of the purified sample through a micro-pipette tip propelled by a pump and this aliquot deposited over each vessel to run PCR test. The power rating of the insert is about 1000 W. A volume of the insert is sized to fit within a galley ARINC size between 4 and 6. The insert can operate independently of the cabin pressure ranges or other environmental conditions in the aircraft. The insert can be inserted within a galley monument for a cabin area of an aircraft, where the monument includes, a first monument stack, a second monument stack adjacent to the first monument stack, a tray within the second monument stack to secure the insert.

The connectivity module can be connected to a cockpit of an aircraft. The power module can be connected to an aircraft power source. The insert can be integrated within a monument stack, or the insert can be removably attached to a monument stack.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is an isometric view of an insert for PCR testing according to the disclosure; and

FIG. 2 is a schematic view of the modules of the insert.

### DETAILED DESCRIPTION

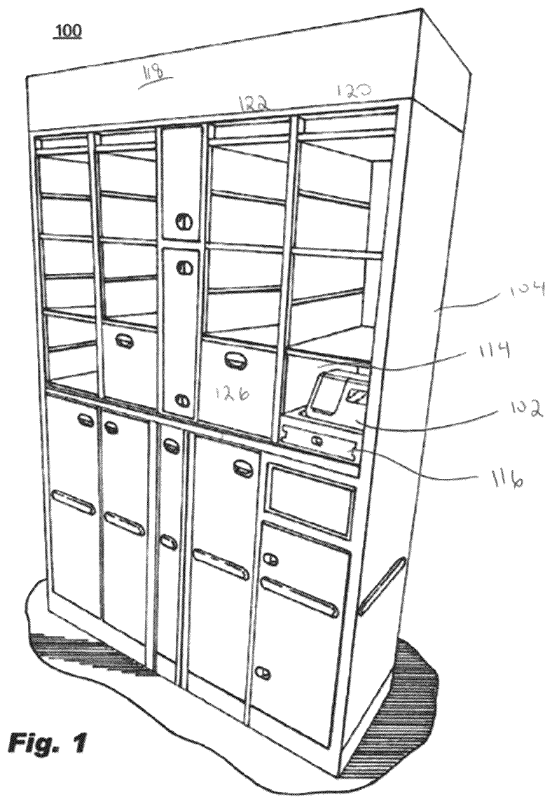

Reference will now be made to the drawings wherein like reference numerals identify similar structural features or aspects of the subject disclosure. For purposes of explanation and illustration, and not limitation, a partial view of an exemplary embodiment of a system in accordance with the disclosure is shown in FIG. 1. The system described below is used to test a bulk sample, representative of all passengers on the aircraft and to test it to provide a bulk screening of the aircraft prior to arrival of the aircraft at a destination.

FIG. 1 shows a modular aircraft galley insert 100 for performing thermal cycling for PCR testing. A storage space 114 is located within the housing 102 for storing for consumables and waste. The waste generated is not medical waste. The consumables are inert, water based, and safe for the environmental. A second storage space 116 can be present for storing the tested sample after a test is complete. The modular aircraft galley insert 100 is inserted within a galley monument 118. The galley monument 118 includes a first monument stack 120, a second monument 122 stack adjacent to the first monument stack 120, and a tray 126 within the second monument stack 124 to secure the modular aircraft galley insert 100.

The power rating of the modular aircraft galley insert 100 is about 1000 W. A volume of the insert is sized to fit within a galley ARINC size between 4 and 6. The insert can operate independently of the cabin pressure ranges or other environmental conditions in the aircraft.

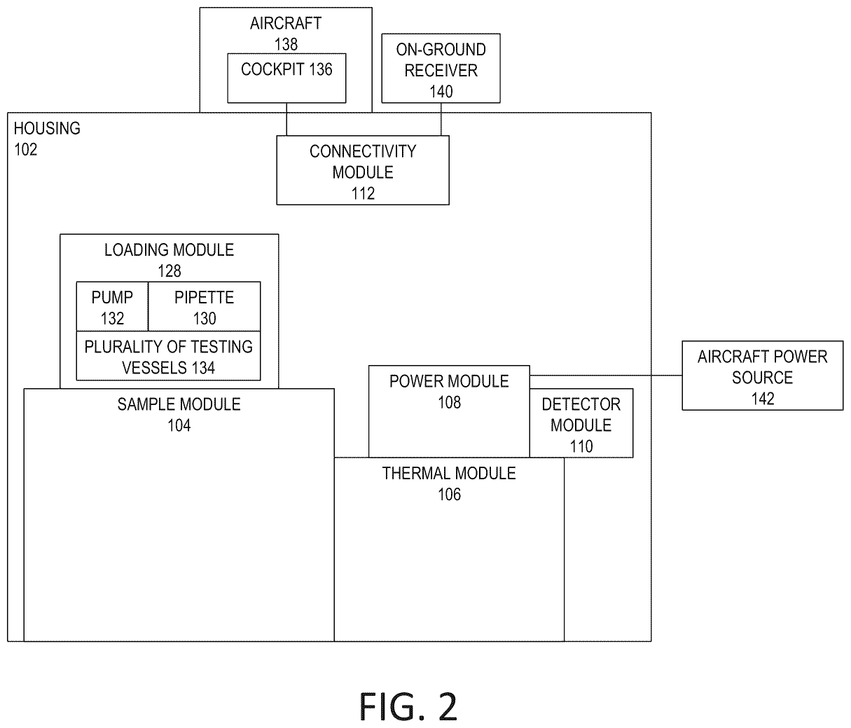

Referring to FIG. 2, the modular aircraft galley insert 100 includes a housing 102 that includes a sample module 104 for receiving a sample to be tested, a thermal module 106 thermally connected to the sample module 104 for cycling temperatures of the sample to be tested, a power module 108 for providing power to the thermal module, a detector module 110 configured to analyze the sample and connected to receive power from the power module 108, and a connectivity module 112 for communicating results of a test from the detector module 110 to a remote location with respect to the housing 102. The housing 102 may further include a second storage space for storing the tested sample. In that regard, the housing module may include a loading module 128 configured to transfer a portion of the purified sample through a pipette 130 propelled by a pump 132 and this aliquot deposited over a plurality of testing vessels 134 to run a PCR test. The connectivity module 112 is be connected to a cockpit 136 of an aircraft 138 in order to relay the results of the test to the crew and pilots. It is also considered that the connectivity module is connected to communicate to a destination or origin airport, i.e., an on-ground receiver 140, to relay the results of the test. The power module 108 can be connected to an aircraft power source 142.

The reagents that are present within the modular aircraft galley insert 100, being ready to use, are mixed, and stable at room temperatures. Reagents will be pathogen-specific, that is, a determined set of reagents for each specific pathogen to be detected. Protocols for running programs are programed in the unit for PCR cycling: a few examples are the setup Temperatures of the specific thermal cycle, timing or number of cycles; in the case of the thermal module.

The methods and systems of the present disclosure, as described above and shown in the drawings, provide for an improved bulk data and analysis of passenger pathogens on an aircraft. The system provides a more efficient primary health controls at airports, helps identify at early stages emerging pathogens and origin of outbreaks, and empower their scientific research (either academic and/or commercial/institutional). While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. An aircraft, the aircraft comprising:
a modular aircraft galley;
a sample; and
an insert positioned within the modular aircraft galley, the insert configured for performing thermal cycling for polymerase chain reaction (PCR) testing on the sample, the insert configured to:
receive the sample;
cycle temperatures of the sample;
analyze the sample; and
communicate results of the analysis of the sample through a connectivity module to a remote location.

2. The aircraft of claim 1, wherein the sample is from consumables and waste materials from passengers on the aircraft.

3. The aircraft of claim 2, wherein the sample is sampled from the consumables and the waste materials and transferred through a pipette tip of a pipette for testing purposes.

4. The aircraft of claim 3, wherein the insert comprises a pump configured to transfer the sample through the pipette and the pipette tip to a plurality of testing vessels configured to receive the sample.

5. The aircraft of claim 1, wherein a power rating of the insert is approximately 1000 W.

6. The aircraft of claim 1, wherein the insert is configured to operate independently of cabin pressure ranges or other environmental conditions in the aircraft.

7. The aircraft of claim 1, wherein the insert is inserted within a galley monument for a cabin area of the aircraft, where the galley monument includes:
a first monument stack;
a second monument stack adjacent to the first monument stack; and
a tray within the second monument stack to secure the insert.

8. The aircraft of claim 1, wherein the connectivity module is connected to a cockpit of the aircraft.

9. The aircraft of claim 1, wherein the connectivity module is connected to an on-ground-receiver.

10. The aircraft of claim 1, wherein the insert is integrated within a monument stack.

11. The aircraft of claim 1, wherein the insert is removably attached to a monument stack.
